(19) United States
(12) Patent Application Publication
(10) Pub. No.: US 2012/0116168 A1
(43) Pub. Date: May 10, 2012

(54) Method and device for flushing during endoscopic surgery

(76) Inventors: Anders MÖLLSTAM, Saltsjo-Boo (SE); Sven MILTON, Vikingstad (SE)

(21) Appl. No.: 13/199,877

(22) Filed: Sep. 12, 2011

Related U.S. Application Data
(60) Provisional application No. 61/403,294, filed on Sep. 13, 2010.

(51) Int. Cl. A61B 1/015

(52) U.S. Cl. 600/155

(57) ABSTRACT

A device and method are provided for flushing during endoscopic surgery to solve the problem of debris in the operating cavity. Included is the ability of reversing the inflow and outflow channels of the endoscope to make possible the beneficial cleaning of the ports of the endoscope from debris and e.g. bone particles without a need for removal of the optical instrument during such flushing procedure and consequently avoiding loss of pressure in the body cavity and spillage. Increased flushing occurs by providing a pressure-holding valve in the proximal opening of the double-channel endoscope, where the optical instrument is inserted, making it possible to remove the optical instrument and then enable the full channel of the endoscope sheath to be used for efficient evacuation of debris without loss of pressure in the operating cavity or spillage on the floor.

### CROSS-REFERENCE TO RELATED APPLICATIONS

This application claims priority from U.S. Provisional Patent Application Ser. No. 61/403,294 filed Sep. 13, 2010.

### BACKGROUND OF THE INVENTION

1. Field of the Invention

This invention relates to the medical field of endoscopic surgery, and in particular a method and device for flushing of liquid in the endoscope channels, inlet and outlets, over the optics and in the surgical cavity as a whole.

2. Description of the Related Art

Endoscopic surgery is performed within the cavities of the human body. A small hole may be created in the skin of the patient and an optical instrument, the endoscope, is positioned in the cavity, or the endoscope can be inserted through a natural entry into the body of the patient. The endoscope can consist of a rigid or flexible tube having channels for the optical instrument, light, fluid, etc. depending on the endoscopy application. Specific applications of this procedure include: laparascopy, enteroscopy, colonoscopy, sigmoidoscopy, proctoscopy, cytoscopy, arthroscopy, etc. The endoscope encompasses an optical instrument for viewing, i.e. rigid scope or a rod lens scope, which is put into a tube assembly, commonly named a sheath, for the optical instrument. The sheath may also have a channel for irrigation of the body cavity. The primary purpose of the irrigation is to distend the body cavity by pressure of the irrigation liquid, and thus enable viewing with the endoscope. Further, the pressurization of the body cavity reduces bleeding from blood vessels breaking as a result of the surgical procedure.

In general the advantage with endoscopic surgery is that it is mainly a less invasive type of operation, which is subject to fewer post-operative risks and after-effects than conventional surgery, and is therefore increasing in popularity in several medical branches. The patient's hospital stay is reduced, which allows endoscopic surgery to be performed at smaller medical centers.

For use in urology, the endoscope is called a cystoscope. The inside of the urinary bladder is examined after positioning the cystoscope in the urethra. Surgical instruments can be introduced in working channel(s) of the endoscope. With cystoscopes such instruments may for instance be electrosurgical tools used to dissect the prostate gland.

In orthopedic applications, or arthroscopy, endoscopy is applied in surgery of body cavities like knee joints, shoulders, hip, elbow etc. In general, all locations for endoscopy are hereinafter referred to as the body cavity.

In arthroscopy, surgical tools such as forceps, “duckbills” and powered tools such as shavers, rotating burrs or electrosurgical tools can be introduced through a second portal into the body cavity. The use of tools causes tissue particles of various sizes to float around in the liquid in the body cavity. These obstruct the view of the operating area and must be removed. When using an arthroscope sheath limited to an irrigation channel, the removal of debris from the body cavity is most often made through a third portal, e.g. the cannula port. This cannula port is connected to a suction source, normally a peristaltic pump. This arrangement allows for a continuous replacement of the liquid as the pump slowly irrigates the body cavity via the irrigation channel of the arthroscope sheath and aspirates via the cannula.

When using surgical tools i.e. shavers or electrosurgical electrodes, debris, and other smaller and larger particles in the rinsing liquid coming from the surgical cavity usually disrupt the view of the operator and depending on the size, even block the drainage from the cavity.

Today the above-mentioned problem is solved by, for example, removing the optical instrument (fiber optic bundles, fiber-optic glass or plastic rods, etc) from the endoscope sheath to allow for draining of irrigation liquid in the scope channels and in the surgical cavity as a whole. This is associated with several problems such as loss of pressure in the surgical cavity and thereby possible bleeding from damaged vessels, collapse of the cavity leading to disrupted visibility and potential discomfort for the patient. Forcing liquids out of the proximal opening of the endoscope sheath leads to uncontrolled flow outside the surgical site including the floor of the operation room. Furthermore there is also an increased risk of contamination of the joint as a relatively big port becomes open to the outside environment when the optical instrument is removed.

Several references disclose different approaches for debris removal and improved visual control through the optics during arthroscopic surgery. WO06014814A discloses a disposable endoscope sheath including an extendable sleeve sized to accommodate an endoscope shaft having a viewing end. The extendable sleeve has a variable length. A distal portion of the sleeve is configured to direct irrigation fluid onto the viewing end of the endoscope to flush surgical debris from the viewing end of the endoscope.

Patent publication EP1161175 discloses an endoscopic instrument having a shaft in which endoscopic optics are disposed. The shaft also serves to supply a cleaning liquid. Flow-influencing means are provided so that the cleaning liquid can reach the front face of the endoscopic optics and eliminate contaminants impairing the view through the endoscopic optics.

U.S. Pat. No. 5,419,309 discloses a cleaning accessory comprising an adaptive and complimentary tubular member for use in conjunction with the endoscope to provide and direct a source of cleansing fluid to the tip of the endoscope so as to enable removal of debris from the optical windows.

U.S. Pat. No. 6,447,446, teaches an endoscope lens cleaning system for removing surgical debris from the objective lens of an endoscope by using irrigation fluid. The system comprises a disposable endoscope sheath and a pump controller switch mounted on said sheath. The system is responsive to a plurality of pre-programmed forward and reverse run times of differing durations.

Another way to remove debris and maintain the pressure in the operating cavity is described in U.S. Pat. No. 7,981,073. This solution, not applicable to all endoscopic procedures, requires that the operating area bears an inflow port for irrigation and an outflow port to which the tubing is connected, wherein the detection of blood cells, red blood cells, hemoglobin and/or debris are performed. The irrigation is normally performed through a single-channel endoscope, for example an arthroscope, which is inserted into a converging comprehensive sheath in which the irrigation fluid is flushed. An outflow port is commonly achieved by insertion of an outflow cannula in a new hole through the patient's skin, to which the evacuation tubing is connected. The drawback with this setup is that the outflow cannula will need an extra port into the operation area. This opens up for postoperative problems like pain from the insertion place, infection in the extra port, etc. To overcome these drawbacks surgeons often refrain from use of the extra port and evacuate the fluids from the operation site by removal of the single-channel arthroscope from the sheath. The pressurized operating area will then be drained in an uncontrolled way through the sheath, which is now open to air. An alternative to this would be to us a double-channel arthroscope, in which both irrigation and evacuation can be performed in a closed system.

Another alternative relates to designing an arthroscope to have two channels for fluids: an inlet and outlet channel. This double-channel arthroscope sheath makes it possible to omit the afore-mentioned cannula and thus save patient pain with one less hole into the body cavity, save operating costs and remove the inconvenience of having the cannula in the way for the surgeon. However when using double-channel arthroscopes, particles of all sizes have to pass through the relatively small inlet ports of the arthroscope sheath. With time these ports become clogged. The removal of debris in the liquid coming out from the surgical site sometimes becomes impossible as the viscosity of the debris or the size of particles in the debris does not allow for a flow through the now clogged outlet channel. In such situations the normal procedure again is to remove the optical instrument from the arthroscope sheath to allow for removal of debris by flushing irrigation liquid in the scope channels and in the surgical cavity as a whole. The lumen of the port of the sheath where the optical instrument normally is positioned is much larger than the lumen of the afore-mentioned outlet channel.

This maneuver of removing the optical instrument is associated with several problems, all of which are similar to the same situation when using a single-channel arthroscope. As the liquid emerges from the body cavity the pressure in the body cavity decreases which might result in bleeding and collapse of the cavity, leading to disrupted visibility within the operation area as well as post-surgery trauma. Replacing the optical instrument in the arthroscope sheath takes time and is an impediment to the procedure because of the urgent need of readjusting the pressure and body cavity distension lost under the aforementioned maneuver.

Further, risk of contamination of the operating staff is elevated as liquids are emerging from the opening of the arthroscope sheath, leading to uncontrolled spillage of irrigation liquids, blood and tissue outside the body cavity including the floor of the operation room. Therefore it is quite common to see the operation staff wearing rubber boots when performing such surgical procedures. Also as a relatively big port become open to the outside air there is a potential risk of contamination of the body cavity.

In this regard U.S. Pat. No. 5,400,767 discloses a device for cleaning the objective lens of an endoscope, without the removal of the endoscope from the body cavity. The device consists of a tube, containing the shaft of the endoscope. At one end of the tube, there is a ridge that can direct a flow of fluid within the tube onto the objective lens of the endoscope shaft. At the other end of the tube there is a means of making a seal, such as a flexible O-ring, that prevents or reduces the leakage of air and/or fluid between the tube and the inserted shaft of the endoscope. During operation, whenever the objective lens at the end of the telescope shaft becomes soiled or obscured, fluid is injected through the aperture in the tube so that it flows between the endoscope shaft and the inner wall of the tube until it reaches the ridge which directs the fluid over the objective lens.

Kiehn et al in US published patent application No. 2005/0085692 discloses a double-channel endoscope with a shaft comprising an outer tube and inside it an inner tube. The inner tube contains an endoscope unit formed by an endoscope tube in which an instrument tube as well as imaging optics have being inserted. This kind of endoscope is commonly used for transurethral surgery in which the rotational position of the surgical instrument relative to the field of surgery frequently must be changed. In order to allow a quick exchange of the endoscope unit during an operation without having to remove the entire endoscope, the endoscope unit, the endoscope tube and the imaging optics are connected with the inner tube in a manner locked against rotation relative to each other. There is provision in this reference of a sealing system for sealing the proximal end of the instrument tube using a rubber-sealing cap with a central hole. The sealing system may comprise two sealing units, with the first sealing unit providing sealing when an instrument is inserted and the second sealing unit providing sealing when no instrument is inserted. The two sealing units are preferably arranged behind each other. This sealing system has the disadvantage that it does not prevent pressure loss when the endoscope unit is removed from the entire endoscope. The sealing system can to some extent protect the patient from infection, but it does not repress the irrigation pressure in the surgical cavity—resulting in leakage of irrigation fluid and subsequent pressure loss. When this sealing system is closed, it obstructs the passage to remove debris via the proximal end of the endoscope arrangement. There is no possibility of removing debris from the surgical cavity when using this type the endoscope.

Recent publications go a bit further by providing both means for cleaning of the endoscope optical surface and means for adjustment of the pressure in the body cavity, which is an essential parameter required in this kind of surgical operation. For example, U.S. Pat. No. 7,341,556 relates to an instrument that includes a gas nozzle supplying a gas jet onto the optical surface under high pressure. A suction pump of an ejection type is arranged in the instrument's handle and can be used also for the removal of abundant secretions or debris. The gas jet and the suction pump, along with control means, form the system for pressure control within the patient's cavity.

U.S. Pat. No. 7,249,602, on the other hand, relates to a surgical endoscopic cutting device in which an outlet for discharging minor parts of fluids and debris; and a further outlet, for discharging substantially only fluid have been provided. The pressure inside the body cavity is regulated by metering the quantity of fluid that moves through the further outlet formed by an insertion tube fitted around the endoscopic device.

Another example comes from U.S. Pat. No. 7,150,713 disclosing an endoscope with an inner portion and a sheath surrounding the inner portion. The inner portion defines an operative channel and an optical channel. The operative channel provides a path for fluid to or from the body site. The sheath defines a pressure-sensing channel and a fluid channel, wherein the pressure-sensing channel may be spatially segregated from the operative channel, the fluid channel, and the optical channel.

In the afore-mentioned systems the methods and devices are limited to handling small particles of debris obtained i.e. after using specially designed cutting devices. The debris that is not aspirated due to temporary restrictions in the aspiration will not be evacuated, but start to float around in the surgical cavity. It is normal during an endoscopic procedure that debris is formed as detachments of the inside of the surgical cavity. With the systems of the prior art, debris is not removed when the cutting device is inactive. If debris needs to be removed, then the cutting device will have to be removed from the endoscope, resulting in pressure drop in the surgical cavity.

Accordingly, there is a need for new and more efficient products and methods for removing debris and rinsing the arthroscope channels: inlet and outlet, the optics and the surgical cavity as a whole, while at the same time avoiding the disadvantage of the products in the prior art with regard to the maintenance of a constant volume and pressure in the body cavity at all times during the operation process. The present invention satisfies those needs. Other objects and advantages will be more fully apparent from the following disclosure.

### SUMMARY OF THE INVENTION

During endoscopic surgery when using surgical tools i.e. shavers or electrosurgical electrodes, debris and other smaller and larger particles in the rinsing liquid coming from the surgical cavity usually disrupt the view of the operator and sometimes also block the drainage from the cavity.

The invention herein solves the above-mentioned problem of debris in the operating cavity in several ways. One basic solution is the introduction of an inflow and an outflow channel, and the ability of reversing the inflow and outflow channels of the endoscope. This make it possible to beneficially clean the aspiration ports of the endoscope from debris and e.g. bone particles without a need for removal of the optical instrument during such flushing procedure and consequently avoiding loss of pressure in the body cavity and spillage.

The present invention further makes it possible to increase the flushing by providing a pressure-holding valve in the proximal opening of the double-channel endoscope, where the optical instrument is inserted, making it possible to remove the optical instrument while utilizing the reversed flow in the endoscope, allowing the full channel for the optical instrument in the sheath to be used for efficient evacuation of debris without loss of pressure and volume in the operating cavity or spillage on the floor.

### BRIEF DESCRIPTION OF THE DRAWINGS

The foregoing summary, as well as the following detailed description of the invention, will be better understood when read in conjunction with the appended drawings. It should be understood, however, that the invention is not limited to the precise arrangements and instrumentalities shown.

FIG. 1 shows the double-flow endoscope positioned in a body cavity.

FIG. 2 shows the double endoscope sheath with reversing valve and a rigid rod lens scope. The rigid rod lens scope is shown at the bottom of the figure.

FIG. 3 shows the assembling of the double-flow endoscope. The rigid rod lens scope is shown at the bottom of the figure.

FIG. 4 shows an example of a setup for a surgical procedure with double arthroscopy pump.

FIG. 5 shows an example of a setup for a surgical procedure with double arthroscopy pump. The reversing valve is fitted on the pump.

FIG. 6 shows the assembled double-flow endoscope with reversed flow indicated.

FIG. 7 shows the assembled double-flow endoscope with the pressure holding valve. FIG. 7A shows a cross-sectional view of an endoscope sheath.

FIGS. 8A-8F are schematic views of the pressure holding valve in closed position (FIGS. 8A-8C) and in open position (FIGS. 8D-8F).

FIG. 9 shows the assembled double-flow endoscope with the pressure holding valve and nominal flow indicated.

FIG. 10 shows the assembled double-flow endoscope with reversed flow indicated and the optical instrument removed for evacuation of debris. FIG. 10A is a cross-sectional view of the endoscope sheath.

FIG. 11 is graph showing pressure and volume variation with time during a shoulder surgical procedure when using an arthroscope.

FIG. 12 is graph showing pressure and volume variation with time during a shoulder surgical procedure when using the double-flow arthroscope of the invention.

FIG. 13 is a graph showing the sequential pressure and volume variation with time during a urological surgical procedure when using a cystoscope.

FIG. 14 is graph showing the pressure and volume variation with time during a bladder surgical procedure when using the double-flow cystoscope of the invention.

### DETAILED DESCRIPTION OF THE INVENTION AND PREFERRED EMBODIMENTS THEREOF

During endoscopic surgical procedures, a surgical site such as a knee joint, shoulder joint or other cavity in the body of a human or animal is viewed with an endoscope. Further in this patent application, the surgical site for an endoscopic procedure is referred to as the body cavity. The body cavity is irrigated with a clear liquid by means of a pump. This pump is further in this patent application referred to as an irrigation pump. The clear liquid is as a rule saline, and the pump is usually a peristaltic roller type pump.

In a first embodiment, the invention allows for a more efficient rinsing of the endoscope channels of debris by using a solution of reversing both the inflow and the outflow channels of a double channel endoscope. Accordingly, the outflow or outlet channel of the endoscope is used as inflow channel for flushing irrigation fluid into the surgical cavity. Once the inflowing liquid in the channel previously used for outflow has cleared the clogged small ports of the endoscope sheath, the in- and outflow channels are used normally again, and the nominal flow pattern is resumed.

The arrangement to reverse the flow of the outflow duct to inflow is accomplished by a control valve arrangement or equivalent fluid control mechanism, for instance pinch mechanisms or pistons applying pinching action on soft tubing. These are further referred to as the reversing valve, which can be part of the endoscope sheath construction, but can also be adjacent to the sheath or even away from the arthroscope sheath. In the latter example, the reversing valve may be fitted on the panel of the pump system used for irrigation and aspiration. The reversing valve may control the liquid with a valve stem, moving door (“lock”) part, control flap(s) or by sliding action in relation to each other of reversing valve parts or similar flow switch function. The reversing valve may be operated manually or powered with an actuator, motor or similar driver.

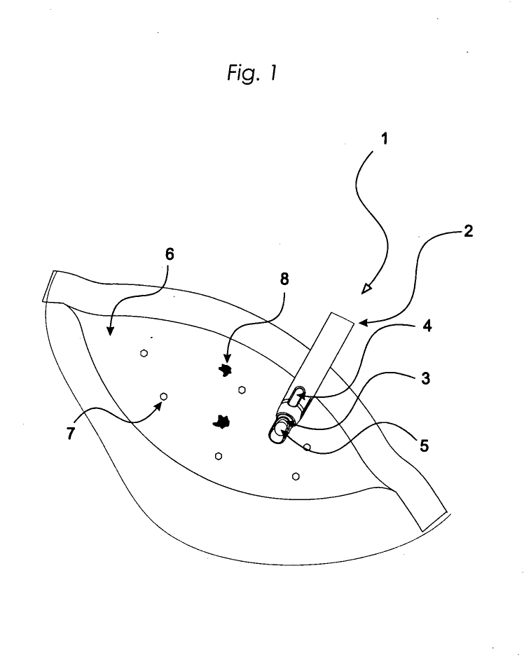

FIG. 1 shows a view of the double-flow endoscope according to the present invention. The double-flow endoscope (1) (only distal end shown) comprises the double-flow endoscope sheath (2) and the optical instrument i.e. the rigid rod lens scope (5). A port (3) for an inner channel and port (4) for outer channel are the liquid communication ports to the body cavity (6). Small pieces of debris (7) as well as large pieces of debris (8) are floating around in the body cavity (6). Surgical tools that have caused the debris are not depicted here. The body cavity (6) can be a joint, bladder, uterus etc.

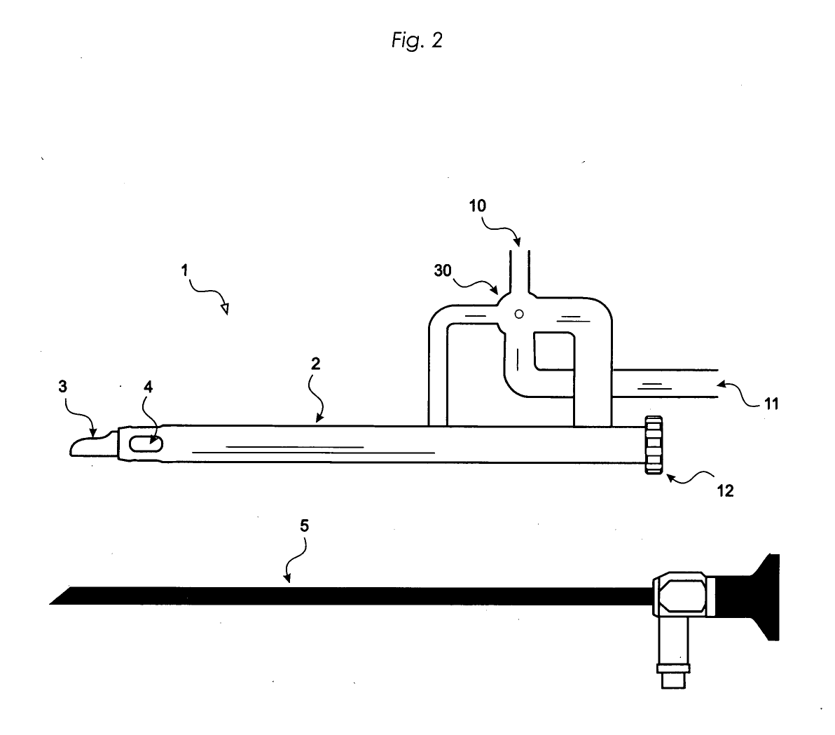

FIG. 2 shows an overview of the double-flow endoscope (1) comprising reversing valve (30), optical instrument (5) and double-flow endoscope sheath (2). Together these make up the double-flow endoscope (1), with features further described below. Irrigation liquid is fed from an irrigation pump or directly from a bag of saline to input port (10) of reversing valve (30), and the waste liquid aspirated from the body cavity (6) emerges from output port (11). The inner channel port (3) and outer channel port (4) of the endoscope sheath (2) are shown, as well as an adaptor (12). The adaptor (12) is fitted to the endoscope sheath (2) by means of threads, a bayonet coupling or similar locking mechanism. The reversing valve (30) may be an integral part of the double-flow endoscope sheath (2).

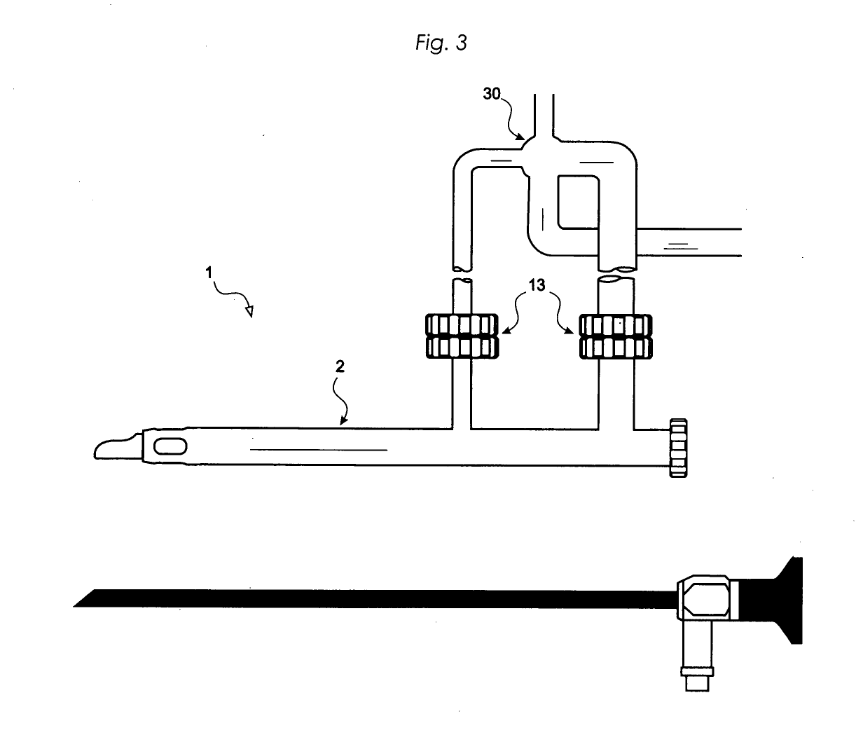

FIG. 3 shows the double-flow endoscope (1) with optional extension connectors (13). The extension connectors (13) make it possible to detach the reversing valve (30) from the double-flow endoscope sheath (2). The use of extension connectors (13) makes the reversing valve (30) a non- integral part of the double-flow endoscope sheath (2). This physical separation between the reversing valve (30) and the endoscope sheath (2) can be advantageous for cleaning purposes and allows personnel other than the surgeon to remotely operate the system.

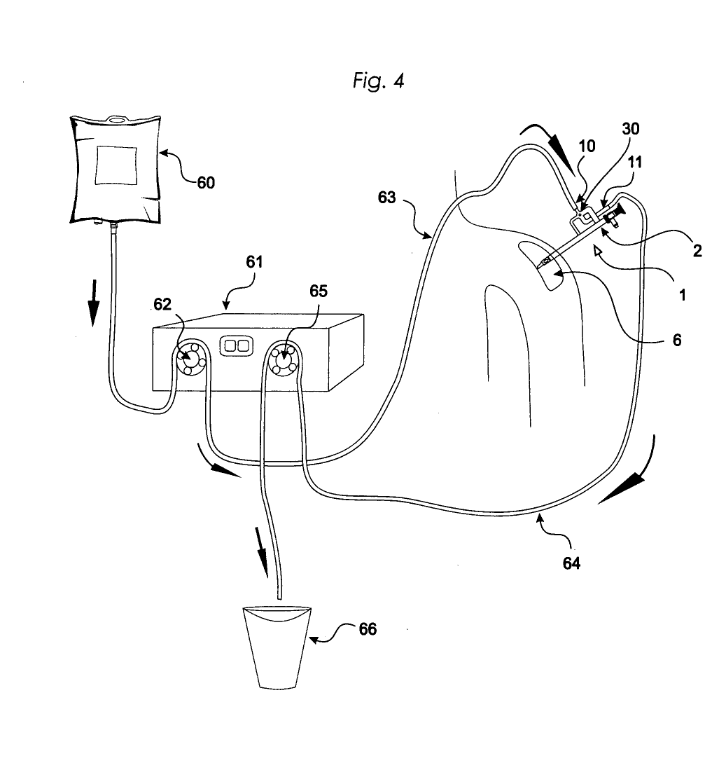

FIG. 4 shows an example of a typical surgical setup for a shoulder surgery procedure while utilizing the double-flow endoscope (1). An irrigation liquid bag (60) feeds irrigation liquid to a double pump (61) for arthroscopy. The double pump (61) for arthroscopy has an irrigation pump (62) that generates pressure and flow into an irrigation tubing (63), connected to the input port (10) of the reversing valve (30). The reversing valve (30) is fitted directly to the endoscope sheath (2). The double-flow endoscope (1) is fitted in the body cavity (6), in this case a shoulder. Liquid emerging from the double-flow endoscope (1) leaves output port (11) of reversing valve (30). The liquid is further transported via the aspiration tubing (64) by the aspiration pump (65) of the double pump (61) into waste bucket (66).

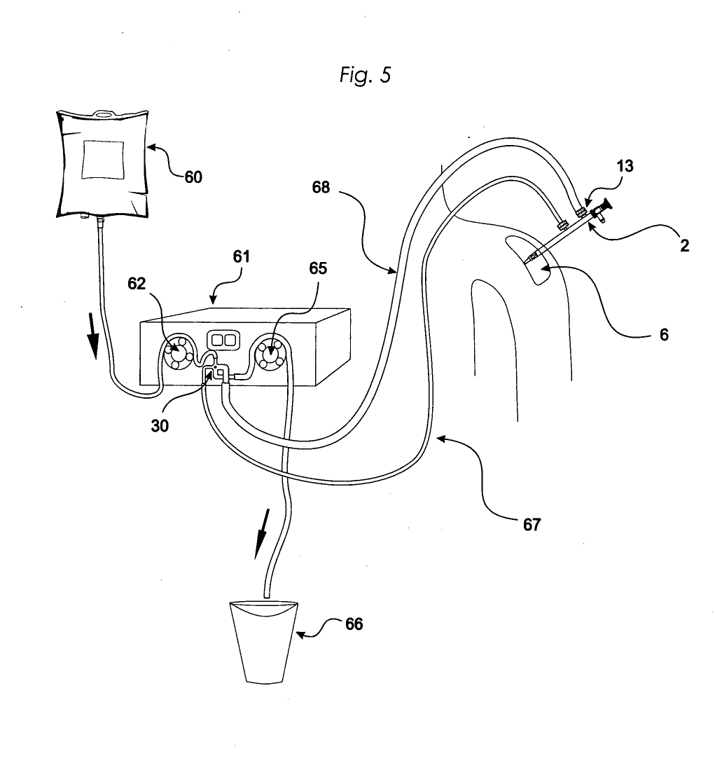

FIG. 5 shows another example of the surgical setup for a shoulder surgery procedure while utilizing the double-flow endoscope (1). In this example, the reversing valve (30) is fitted on the front panel of the arthroscopy pump. The advantage of this variety is that the reversing valve (30) can be remotely controlled by an actuator fitted in or by the pump. The valve actuation can be remotely controlled by use of a foot control for the pump, or automatically for instance by timer control. The irrigation liquid is fed from the reversing valve (30) to the double-flow endoscope (1) via either tubing 67 or 68, depending on the setting of the reversing valve (30). The tubing 67 and 68 are connected to the double-flow endoscope sheath (2) via the extension connectors (13).

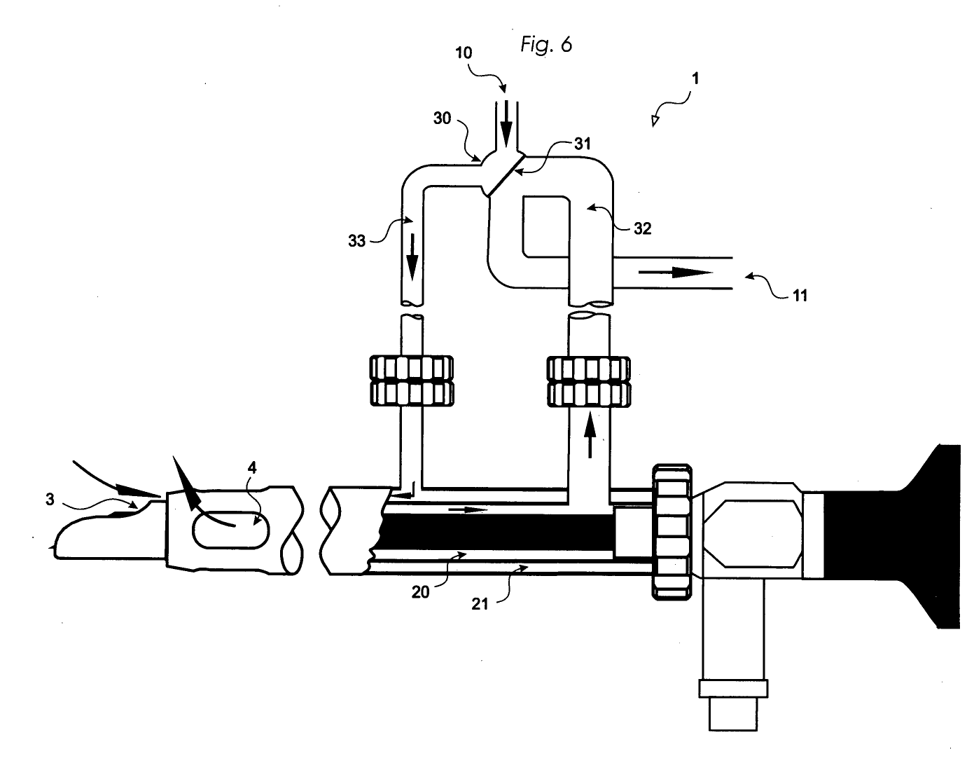

FIG. 6 shows a first preferred mode of use of the double-flow endoscope (1) of the present invention. Irrigation liquid is administered from a pump to input port (10) of reversing valve (30) with a flow switch (31) set to direct the flow towards a second channel (33) leading to outer channel (21) of double-flow sheath (2). The outer channel (21) directs the liquid towards the body cavity (6) through port (4) for the outer channel (21). From the body cavity (6) the port (3) for inner channel (20) leads the liquid via inner channel (20) to a first channel (32) to the reversing valve (30). The flow switch (31) is set to direct the flow towards output port (11). Flow is indicated with flow arrows. First channel (32) and second channel (33) are tubes that connect from the reversing valve (30) to the endoscope (1) as shown and discussed herein.

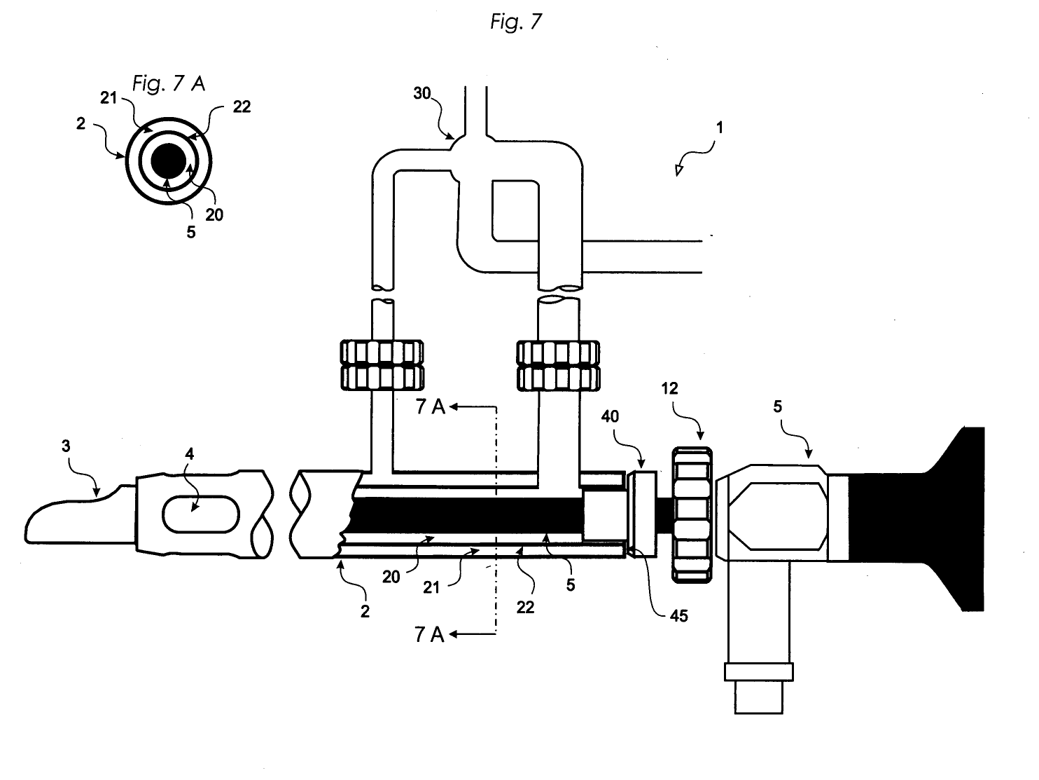

An alternative embodiment of the present invention is shown in FIG. 7. The adaptor (12) of the double-flow endoscope (1) is fitted to the endoscope sheath (2) by means of threads, a bayonet coupling or similar locking mechanism. The adaptor (12) holds in place a pressure holding valve (40), located at the proximal end of the endoscope sheath (2). The inner channel (20) is formed between the inner tube (22) and the optical instrument (5). The outer channel (21) is formed between the inner tube (22) and the inside of the endoscope sheath (2). This is also shown for clarity with cross-sectional view 7A. The optical instrument (5), pries the pressure holding valve (40) to the open position. The pressure holding valve (40) seals against the optical instrument (5), the inner tube (22) of the endoscope sheath (2) and even the proximal end of the endoscope sheath (2) at seal (45). When it is mounted, the adaptor (12) presses against pressure holding valve (40) at seal (45).

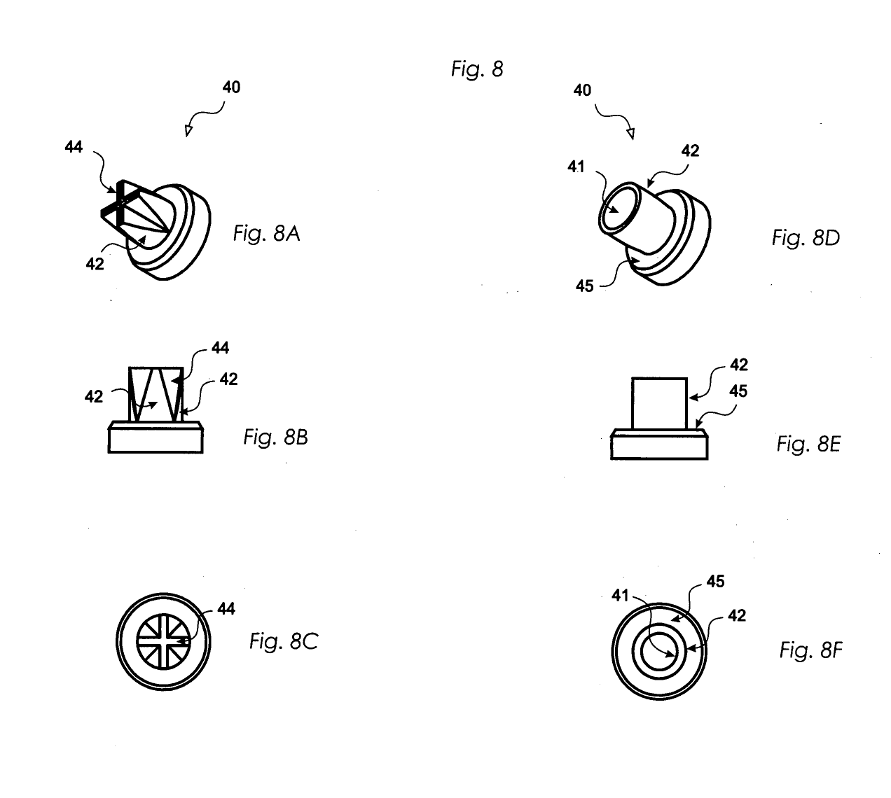

FIGS. 8A-8F show an example of the pressure holding valve (40). It is manufactured in a soft and pliable material e.g. silicone, that has the capability to return to its original shape if deformed. FIGS. 8A-8C depict the valve in the closed position in three projections, which is the relaxed shape. Inner sealing surface (41) seal against the optical instrument (5), and outer sealing surface (42) seal against the double-flow endoscope sheath (2). In the closed state, the valve lips (44) are closed as a result of the material to return to the relaxed shape. FIGS. 8D-8F depict the valve in the open position in three projections. Here, the valve lips (44) are forced open by the optical instrument i.e. a rod lens scope (not shown)—forming a circular shape. A flat surface on the proximal end of the pressure holding valve (40) may optionally form an endoscope end seal (45).

The pressure holding valve (40) can be a custom-designed, one-piece valve that provides reliable flow prevention when closed. This preferably has an inner diameter of 4 mm to be used in 4 mm rod lens scope, 2.7 mm for 2.7 mm rod lens scope, etc. The pressure in the closed state can nominally range from 20-150 mmHg, but in extreme conditions the user may have applied a pressure of over 180 mmHg to a joint. In the case of urinary bladders, the pressure is as a rule less than 50 mmHg, and in all cases the pressure holding valve (40) shall not leak in a closed state.

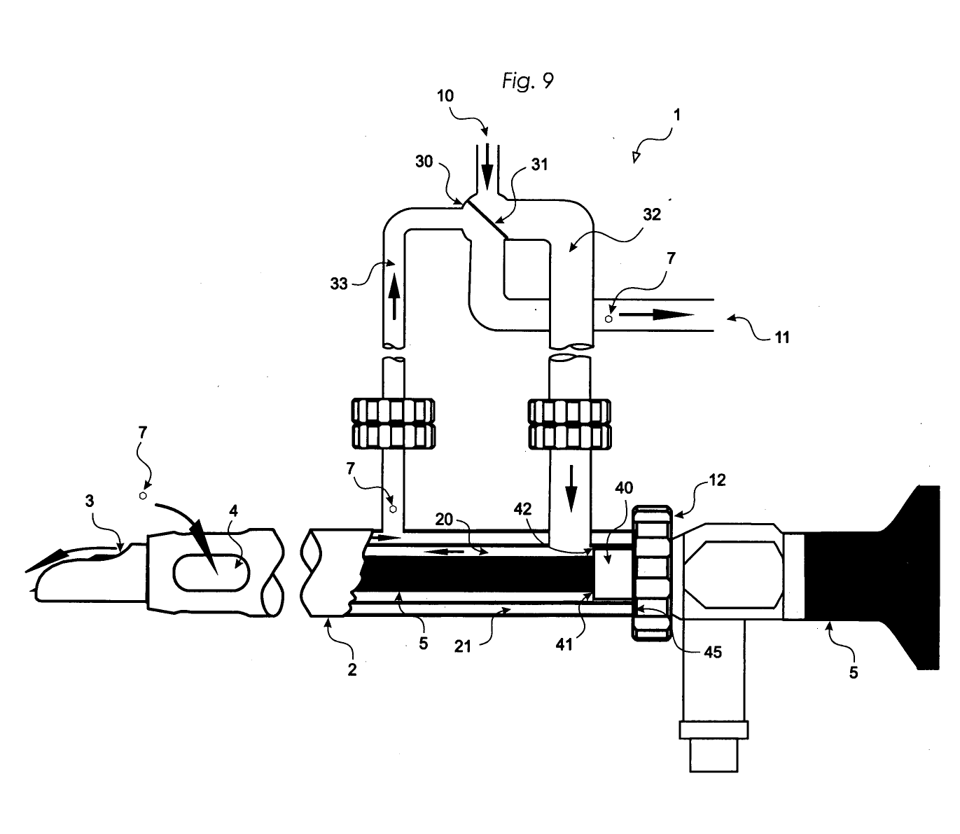

FIG. 9 shows a further preferred mode of use for the double-flow endoscope (1) of the invention herein. Irrigation liquid is administered from a pump to input port (10) of reversing valve (30) with a flow switch (31) set to direct the flow towards a first channel (32) leading to inner channel (20) of double-flow sheath (2). The inner channel (20) directs the liquid towards the body cavity (6) through port (3) for the inner channel (20). From the body cavity (6) the port (4) for outer channel (21) leads the liquid via outer channel (21) to a second channel (33) to the reversing valve (30). The flow switch (31) is set to direct the flow towards output port (11). Flow is indicated with flow arrows. Small pieces of debris (7) can be evacuated. The proximal end of the endoscope sheath (2) is sealed against leakage at the proximal end with the inner sealing surface (41) and the outer sealing surface (42). Sealing is also achieved by the endoscope end seal (45). It is accomplished by the application pressure of the adaptor (12) against the end of the endoscope sheath (2).

The presence of pressure holding valve (40) at the proximal end of the endoscope sheath (2) is essential in those cases when it is necessary to remove the optical instrument (5) from the endoscope sheath (2) to allow for removal of larger debris by flushing irrigation liquid in the scope channels or for changing the optical instrument to for example another magnification or cleaning of the front lens. The automatic pressure holding valve (40) of the invention both stops irrigation liquid from leaving the inner tube (22) at the proximal end; and helps in keeping a constant pressure at the body site. Once the optical instrument (5) is removed, the inner tube (22) can form a low resistance outflow path.

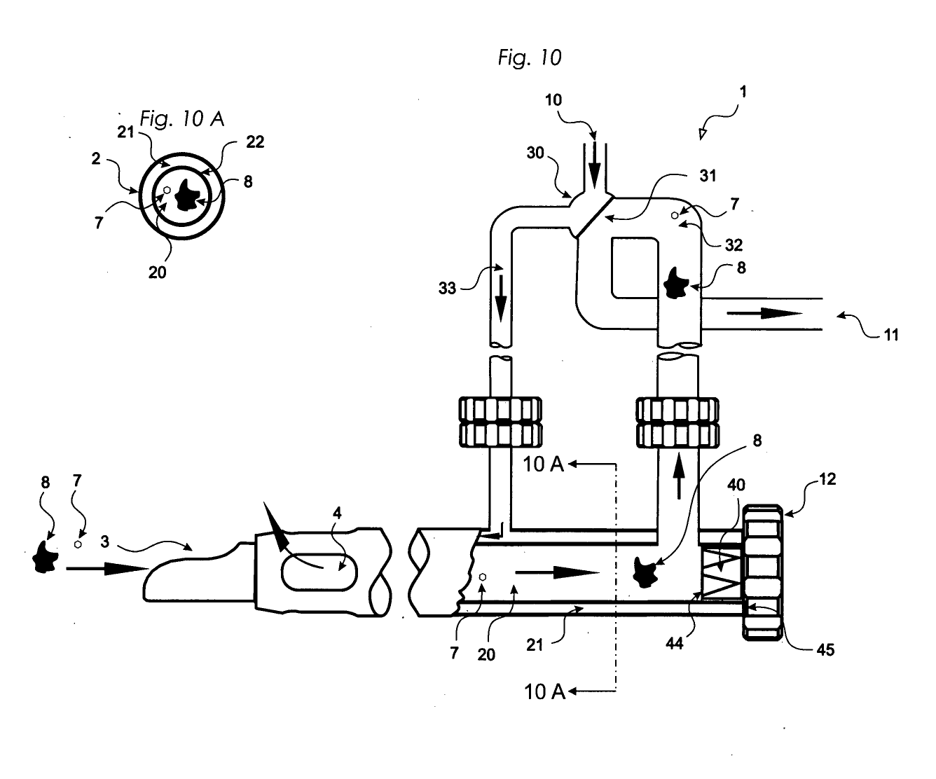

As shown in FIG. 10, the irrigation liquid is administered from a pump to input port (10) of reversing valve (30) with a flow switch (31) set to direct the flow towards a second channel (33) leading to outer channel (21) of double-flow sheath (2). The outer channel (21) directs the liquid towards the body cavity (6) through port (4) for the outer channel (21). From the body cavity (6) the port (3) for inner channel (20) leads the liquid via inner channel (20) to a first channel (32) to the reversing valve (30). The flow switch (31) is set to direct the flow towards output port (11) where both liquid and large pieces of debris (8) can pass. The inner channel (20) has a considerably higher capacity as the optical instrument (5) is removed. Large pieces of debris (8) that were floating around in the body cavity (6) can now be evacuated, as well as small pieces of debris (7). This is also shown for clarity with cross-sectional view 10A. Flow is indicated with flow arrows. The proximal end of the endoscope sheath (2) is sealed against leakage and pressure loss at the proximal end as the valve lips (44) of the pressure holding valve (40) closes. Also, the pressure in the inner channel (20) is acting on the lips (44) pressing these together, forming a tight liquid and pressure seal.

Basically, in this last embodiment with both flow reversal and removal of the optical instrument (5), the small distal ports of the outflow channel of the sheath (2) become irrigation ports and the irrigation channel from which the optical instrument was removed, will serve as a large outflow channel.

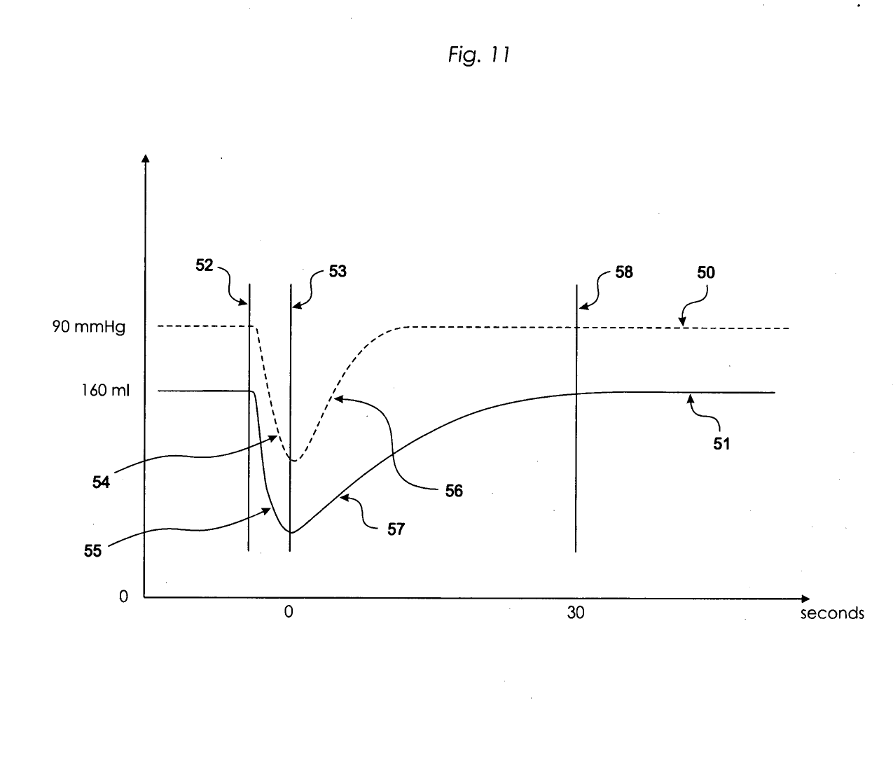

FIG. 11 shows the sequential pressure and volume variation during a shoulder surgical procedure when using an arthroscope. The x-axis represents time in seconds. The dotted line (50) is pressure in the shoulder. The liquid volume in the shoulder is shown by line (51), and this corresponds to the distention of the shoulder. The more the shoulder is distended, the better view. The shoulder is in this example initially pressurized to 90 mmHg, resulting in a volume of 160 ml liquid in the shoulder. The optical instrument is removed from the arthroscope sheath for rinsing of the shoulder. The occurrence in time is shown by the vertical line (52). As liquid emerges from the shoulder, the pressure drops as shown at (54), as a result of liquid escaping from the shoulder via the arthroscope sheath. The liquid volume in the collapsing shoulder is shown to escape by the volumetric drop (55). At a point in time shown by vertical line (53), the optical instrument is reinserted in the arthroscope sheath. This stops the escape of liquid from the shoulder, and pressure is slowly restored as shown at (56). At this point in time (53), the distention volume is very small, and it is very difficult—or impossible—to view the inside of the shoulder for that reason. The liquid volume is slowly restored as indicated at (57). At time point (58) the full inside view of the shoulder is regained. A substantial time has passed between the time interval between reinsertion of the optical instrument (53) and regained distention volume (58). This is typically 30 seconds—in this example—and is a very disturbing interruption of the surgical procedure.

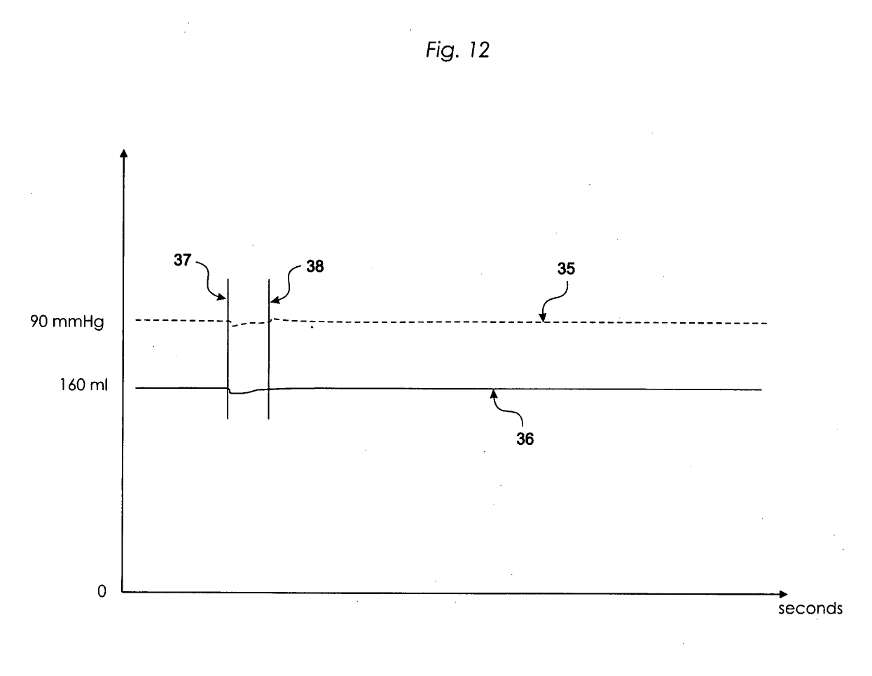

FIG. 12 is a diagram showing the pressure and volume variation during a shoulder surgical procedure when using the double-flow arthroscope of the present invention. The optical instrument is removed at time point (37) and reinserted at (38). Neither significant pressure nor volumetric loss occurs, as shown by pressure curve (35) and volume curve (36). View of the inside of the shoulder is not interrupted. The key beneficial feature is maintenance of distention volume.

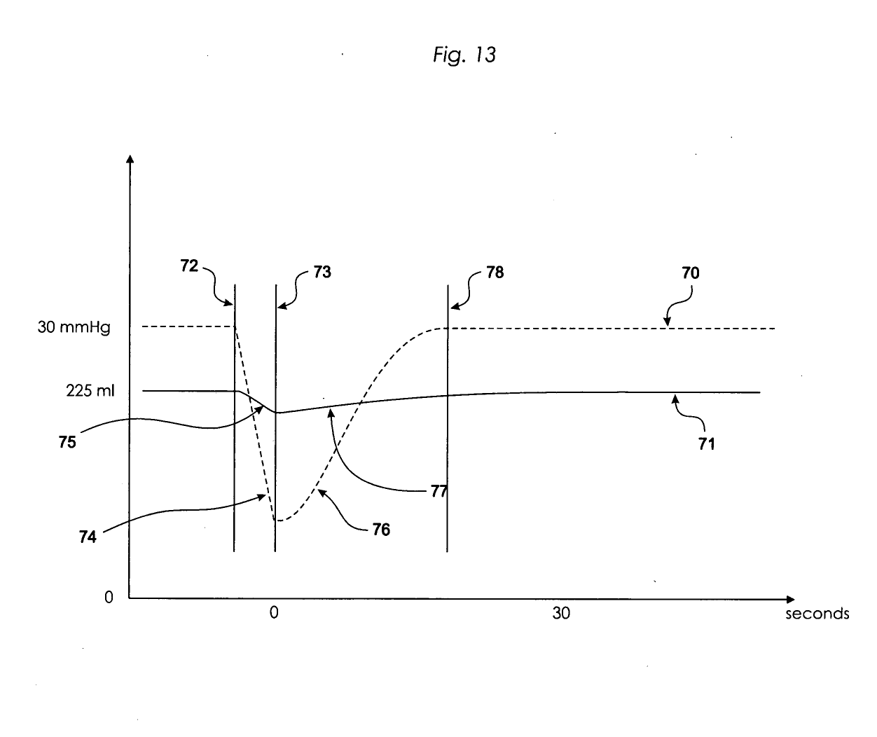

FIG. 13 shows the sequential pressure and volume variation during a urological surgical procedure when using a cystoscope. The x-axis represents time in seconds. The dotted line (70) represents the pressure in the bladder. The liquid volume in the bladder is shown by line (71), and this corresponds to the distention of the bladder. The more the bladder is distended, the better view. The bladder is in this example initially pressurized to 30 mmHg, resulting in a volume of 225 ml liquid in the bladder. The optical instrument is removed from the cystoscope sheath for rinsing of the bladder from debris such as electro-surgically removed shavings of the prostate gland. The occurrence in time is shown by the vertical line (72). As liquid emerges from the bladder, the volume drops as shown at point (75), as a result of liquid escaping from the bladder via the cystoscope sheath. The liquid pressure in the bladder also drops significantly as shown at point (74). In the period of time between vertical line (72) and line (73), the distention volume decreases, and more importantly, pressure drops to an extent where it may cause bleeding of the dense vasculature of the prostate gland. At the time point shown by vertical line (73), the optical instrument is reinserted in the cystoscope sheath. This stops the escape of liquid, and pressure is restored as shown at (76). The liquid volume is slowly restored as indicated at (77). Consequently at time point (78) the pressure of the bladder is regained. A substantial time has passed between the time interval between reinsertion of the optical instrument (73) and regained pressure (70). This is typically 20 seconds—in this example—and is a very unwelcome interruption of the surgical procedure as blood emerges and reduces the view.

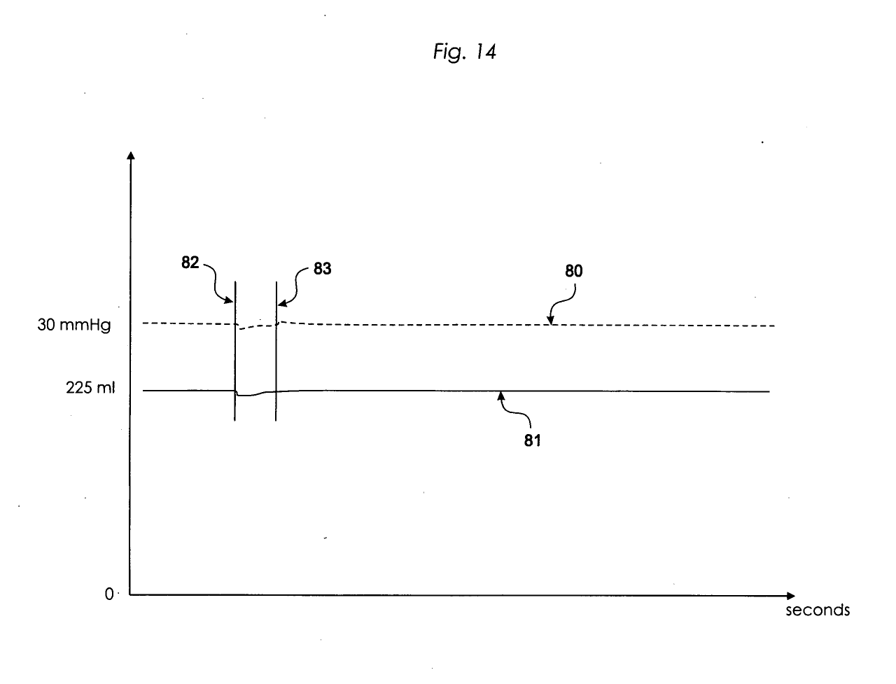

FIG. 14 is a diagram showing the pressure and volume variation during a bladder surgical procedure when using the double-flow cystoscope of the present invention. The optical instrument is removed at time point (82) and reinserted at time point (83). Neither significant pressure nor volumetric loss occurs, as shown by pressure curve (80) and volume curve (81). The key beneficial feature is then that pressure in the bladder is maintained at all times.

With respect to the above description, it is to be realized that the optimum dimensional relationships for the parts of the invention, to include variations in size, materials, shape, form, function and manner of operation, assembly and use, are deemed readily apparent and obvious to one skilled in the art.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A device for removing debris and rinsing endoscope channels during endoscopic surgery of a body cavity, comprising:
a) a double-channel endoscope comprising a double-channel endoscope sheath for insertion of a optical instrument, the double-channel endoscope sheath having a proximal and distal end, having an inflow channel and an outflow channel, and having an inner channel port and an outer channel port;
b) a optical instrument;
c) a device for reversing the inflow and outflow channels of the endoscope, wherein debris may be removed from the body cavity without removal of the endoscope during flushing of the endoscope and body cavity and without loss of pressure in the body cavity and spillage; and
d) a source of irrigation liquid.

2. The device of claim 1, wherein the device for reversing the inflow and outflow channels comprises a reversing valve.

3. The device of claim 2, wherein the reversing valve comprises an input port and an output port.

4. The device of claim 2, wherein the reversing valve is part of the endoscope.

5. The device of claim 2, wherein the reversing valve is fitted on the source of irrigation liquid.

6. The device of claim 1, wherein the source of irrigation liquid comprises a saline bag.

7. The device of claim 1, wherein the source of irrigation liquid comprises an irrigation pump.

8. The device of claim 1, further comprising tubing connecting the source of irrigation liquid to the device for reversing the inflow and outflow channels.

9. The device of claim 1, further comprising a pressure-holding valve at the proximal end of the double-channel endoscope sheath where the optical instrument is inserted, so that the optical instrument may be removed while utilizing the reversed flow in the endoscope, allowing the entire scope channel to be used for efficient evacuation of debris without loss of pressure and volume in the body cavity or spillage on the floor.

10. The device of claim 9, further comprising an inner sealing surface and an outer searing surface for sealing the proximal end of the endoscope sheath against leakage.

11. The device of claim 1, further comprising an irrigation pump for irrigating the body cavity.

12. The device of claim 11, wherein the irrigation pump is a peristaltic roller type pump.

13. The device of claim 1, further comprising extension connectors between the endoscope sheath and the device for reversing the inflow and outflow channels of the endoscope, so that the device for reversing the inflow and outflow channels of the endoscope may be detached from the endoscope sheath.

14. The device of claim 1, further comprising a first channel and a second channel connecting the double-channel endoscope to the device for reversing the inflow and outflow channels of the endoscope.

15. The device of claim 1, further comprising a first channel and a second channel connecting the double-channel endoscope to the device for reversing the inflow and outflow channels of the endoscope, and wherein the device for reversing the inflow and outflow channels comprises a reversing valve comprising an input port and an output port.

16. A method for removing debris and rinsing endoscope channels during endoscopic surgery of a body cavity, comprising:
a) providing the device of claim 1;
b) placing the endoscope in the body cavity; and
c) using the outflow channel of the endoscope as an inflow channel for flushing irrigation fluid into the surgical cavity, followed by using the outflow channel for outflow and the inflow channel for inflow once the inflowing liquid in the channel previously used for outflow has cleared clogged ports of the endoscope.

17. The method of claim 16, wherein irrigation liquid is administered by a pump.

18. The method of claim 16, wherein providing the device of claim 1 further comprises providing a first channel and a second channel connecting the double-channel endoscope to the device for reversing the inflow and outflow channels of the endoscope and wherein the device for reversing the inflow and outflow channels comprises a reversing valve comprising an input port and an output port, the method further comprising administering irrigation liquid from a pump to the input port with a flow switch set to direct the flow toward the second channel leading to the outer channel which directs the liquid toward the body cavity through the outer channel port, with the liquid flowing from the body cavity through the inner channel port through the inner channel to the first channel to the reversing valve.

19. The method of claim 16, wherein providing the device of claim 1 further comprises providing a first channel and a second channel connecting the double-channel endoscope to the device for reversing the inflow and outflow channels of the endoscope and wherein the device for reversing the inflow and outflow channels comprises a reversing valve comprising an input port and an output port, the method further comprising administering irrigation liquid from a pump to the input port with a flow switch set to direct the flow toward the first channel leading to the inner channel which directs the liquid toward the body cavity through the outer channel port through the outer channel to the second channel to the reversing valve.
